(19) United States
(12) Patent Application Publication
(10) Pub. No.: US 2016/0114101 A1
(43) Pub. Date: Apr. 28, 2016

(54) INJECTION DEVICE AND METHOD FOR DILUTING AN INJECTABLE FLUID

(71) Applicant: Allergan, Inc., Irvine, CA (US)

(72) Inventors: Christopher S. Mudd, Fort Worth, TX (US); Mitchell H. Babkes, Santa Clarita, CA (US); Zachary P. Dominguez, Santa Barbara, CA (US); Michael J. Prichard, Calisle, MA (US); Martha E. Llewellyn, Maynard, MA (US); Shaohui Qiu, Belmon, MA (US); Lee F. Powers, Somerville, MA (US); Brett R. Johnson, Roseville, MN (US)

(21) Appl. No.: 14/990,694

(22) Filed: Jan. 7, 2016

Related U.S. Application Data
(63) Continuation of application No. 13/658,357, filed on Oct. 23, 2012.
(60) Provisional application No. 61/561,748, filed on Nov. 18, 2011.

(51) Int. Cl. A61M 5/14; A61M 5/19; A61M 5/172

(52) U.S. Cl. CPC A61M 5/1407; A61M 5/172; A61M 5/19; A61M 2205/33

(57) ABSTRACT

An injection device enables a user to control the dilution ratio of mixed injectable fluid. In one embodiment, the injection device includes a drive unit configured to apply extrusion forces to fluids. In one embodiment, the injection device produces the mixed injectable fluid based on a selected dilution ratio. In one embodiment, the injection device produces the mixed injectable fluid based on selected injection rates.

### RELATED APPLICATIONS

This application is a continuation if U.S. patent application Ser. No. 13/658,357, filed on Oct. 23, 2012, which application claims priority to and the benefit of U.S. Provisional Patent Application No. 61/561,748, filed Nov. 18, 2011, the entire disclosure of each of these documents being incorporated here by this specific reference.

### BACKGROUND

A number of medical and cosmetic applications involve controlled injection of substances into the body.

A medical syringe is a simple piston pump consisting of a plunger that fits tightly in a cylindrical barrel. The plunger can be pulled and pushed along inside the barrel, allowing the syringe to take in and expel a fluid through an orifice at the distal open end of the barrel. The distal end of the syringe is typically fitted with a hypodermic needle to subcutaneously introduce the barrel's fluid into a patient. Surprisingly, other than the materials used to make a syringe, the typical disposable syringes are much the same as the very earliest syringe designs.

Unfortunately, a classic syringe/needle systems are far from optimal for the administration of today's injectable aesthetic compositions. Hydrogel-based dermal fillers can be quite difficult to inject using the conventional syringe/needle system or conventional injection techniques. Many dermal fillers are by their nature highly viscous, thus requiring relatively high extrusion forces, especially when injected through preferred fine gauge needles. Moreover, these materials are typically injected into the face to correct wrinkles, including fine wrinkles as well as other minor defects in skin, and therefore, must be sometimes injected in trace amounts, and always with very high precision. Interestingly, these dermal fillers are commonly introduced into skin using quite standard needle and syringe combinations.

Using a traditional syringe, physicians can be required to supply possibly significant force, which may reduce the practitioner's ability to control the syringe. Further, traditional syringes typically require the user's hand to be placed a significant distance from the site of the injection in order to operate the plunger, which may also lead to inaccuracy.

As an additional complexity, it can be desired to mix fluids prior to injection based on any number of factors such as, for example, the size of a patient's wrinkle. To increase user control of injections and accuracy of mixing injectable fluids, it is desirable to provide users with new types of injection devices. Accordingly, a need exists for further development of injection devices.

### SUMMARY

In one embodiment, injection devices can include: (a) at least one processor; (b) at least one input device operatively coupled to the processor; (c) a first cartridge that defines a first chamber which is configured to contain a first injectable fluid (e.g., a dermal filler); (d) a second cartridge that defines a second chamber which is configured to contain a second injectable fluid (e.g., a phosphate buffered saline); (e) a drive unit operatively coupled to the processor; (f) a mixing unit configured to mix the first injectable fluid and the second injectable fluid; and (g) at least one memory device storing instructions. In operation, the injection device can select a dilution ratio of the first injectable liquid and the second injectable liquid. In one embodiment, the injection device can select the injection ratio based on a user's input. Using the selected dilution ratio, the injection device may produce an injectable mixed fluid by diluting the first injectable liquid with the second injectable liquid. Thereafter, using the drive unit, the injection device extrudes the injectable mixed fluid.

In one embodiment, the drive unit includes gear motors and racks operatively coupled to the gear motors. In this example, the racks are operatively engaged with plungers. In another example, the drive unit includes a pressure source and a pressure regulator.

In one embodiment, the injection device selects an injection rate for the mixed injectable fluid. In this example, the injection device extrudes the injectable mixed fluid based on the selected injection rate. In another example, the injection device selects a first injection rate for the first injectable fluid, and selects a second injection rate for the second injectable fluid.

In some examples, the injection device may be configured to display any of the injection rates. In some examples, the injection device displays information indicating a volume of fluid injected.

Additional features and advantages are described herein, and will be apparent from, the following Detailed Description and Figures.

### BRIEF DESCRIPTION OF THE DRAWINGS

FIGS. 1A and 1B illustrate perspective views of one embodiment of an injection device disclosed herein.

FIGS. 1C and 1D illustrate cross-sectional perspective views of the injection device of FIGS. 1A and 1B, illustrating the drive unit having dual gear motors.

FIG. 1E illustrates a schematic diagram of the injection device of FIGS. 1A and 1B having an electronic configuration, illustrating a processor, a memory device, input devices and output devices.

FIGS. 2A, 2B and 2C illustrate front views of one embodiment of displays of the injection device, illustrating the selection of the dilution ratio and the injection rate.

FIG. 3 illustrates a perspective view of one embodiment of a component, illustrating two cartridges combined into one component.

FIG. 4 illustrates a cross-sectional perspective view of one embodiment of a single cartridge, illustrating the single cartridge having two chambers.

FIG. 5 illustrates a schematic diagram of one embodiment of a single cartridge, illustrating a regulator being used to control the dilution ratio of a combination of fluids.

FIG. 6 illustrates a perspective view of one embodiment of the mixing unit, illustrating the mixing unit having a spiral mixing path.

FIG. 7 illustrates a cross-sectional perspective view of one embodiment of the mixing unit, illustrating the mixing unit having a helical mixing path.

FIG. 8 illustrates a cross-sectional perspective view of one embodiment of the mixing unit, illustrating the mixing unit having corrugated sections.

FIG. 9 illustrates a schematic diagram of one embodiment of the drive unit, illustrating the drive unit having independent dual gear motors.

FIG. 10 illustrates a schematic diagram of one alternative example of the drive unit, illustrating the drive unit having a single gear motor and a transmission.

FIGS. 11A and 11B illustrate schematic diagrams of alternative examples of the drive unit, illustrating the drive unit being a pressure driven system.

FIG. 12 illustrates a schematic diagram of one alternative example of the drive unit, illustrating the drive unit being a hydraulically driven system.

FIG. 13 illustrates a schematic diagram of one alternative example of the drive unit, illustrating the drive unit having a nitinol hydraulically driven system.

FIGS. 14A and 14B illustrate cross-sectional perspective views of one embodiment of the component configuration of the injection device, illustrating the batteries of the injection device being positioned under the cartridges of the injection device.

FIG. 15 illustrates a perspective view of one embodiment of the component configuration of the injection device, illustrating the batteries and motors of the injection device being positioned at the rear section of the injection device.

FIG. 16 illustrates a perspective view of one embodiment of the component configuration of the injection device, illustrating the motors and the cartridges of the injection device forming a diamond shaped cross section by being positioned at the front section.

FIG. 17 illustrates a perspective view of one embodiment of the component configuration of the injection device, illustrating a single motor being positioned under the cartridges at the front section of the injection device.

FIG. 18 illustrates a perspective view of one embodiment of the component configuration of the injection device, illustrating a potentially unobstructed viewing of the cartridges by positioning certain components of the injection device in the rear section of the injection device.

FIG. 19 illustrates a perspective view of one embodiment of the component configuration of the injection device, illustrating the injection device having a compact front section.

FIGS. 20A and 20B illustrate perspective views of one embodiment of the component configuration of the injection device, illustrating the component configuration enabling a user to manipulate the injection device similar to an existing needle and syringe device.

### DETAILED DESCRIPTION

Described herein generally are injection devices including: (a) cartridges configured to contain injectable fluids; (b) a mixing unit configured to mix the injectable fluids to produce an injectable mixed fluid; (c) a control system; and (d) an injection drive mechanism or a drive unit configured to cause: (i) the injectable fluids to mix; and (ii) the injectable mixed fluid to be extruded from the injection device.

In the general operation of one embodiment, before an injection occurs, the injection device can enable a user to select a dilution ratio of a first injectable fluid (e.g., hyaluronic acid (“HA”)) and a second injectable fluid (e.g., phosphate buffered saline (“PBS”)). As the injectable fluids move from their respective chambers towards the needle for extrusion, using a mixing unit, the injection device can dilute the first injectable liquid with the second injectable liquid based on the selected dilution ratio. In one embodiment, the injection device also enables the user to control the rate at which the mixed fluid extrudes from the injection device.

As mentioned above, a number of medical and cosmetic procedures involve the controlled injection of liquids, gels, and other fluids. For instance, procedures involving the injection of botulinum toxin or the injection of dermal fillers, may require highly controlled injections. Using the injection devices and methods disclosed herein, users need not supply some or all the force required to extrude the mixed injectable fluid. The injection devices and methods described herein provide highly controlled injections, by having the injection device: (i) supply the force which extrudes the injectable fluid through the needle; and (ii) extrude the fluid at a user controlled rate and with a user controlled dilution ratio, leaving the user free to concentrate on the injection itself, e.g., positioning of the needle. Additionally, some examples disclosed herein may also provide a more balanced injection device and facilitate injection for a wide variety of hand shapes, sizes and gripping positions.

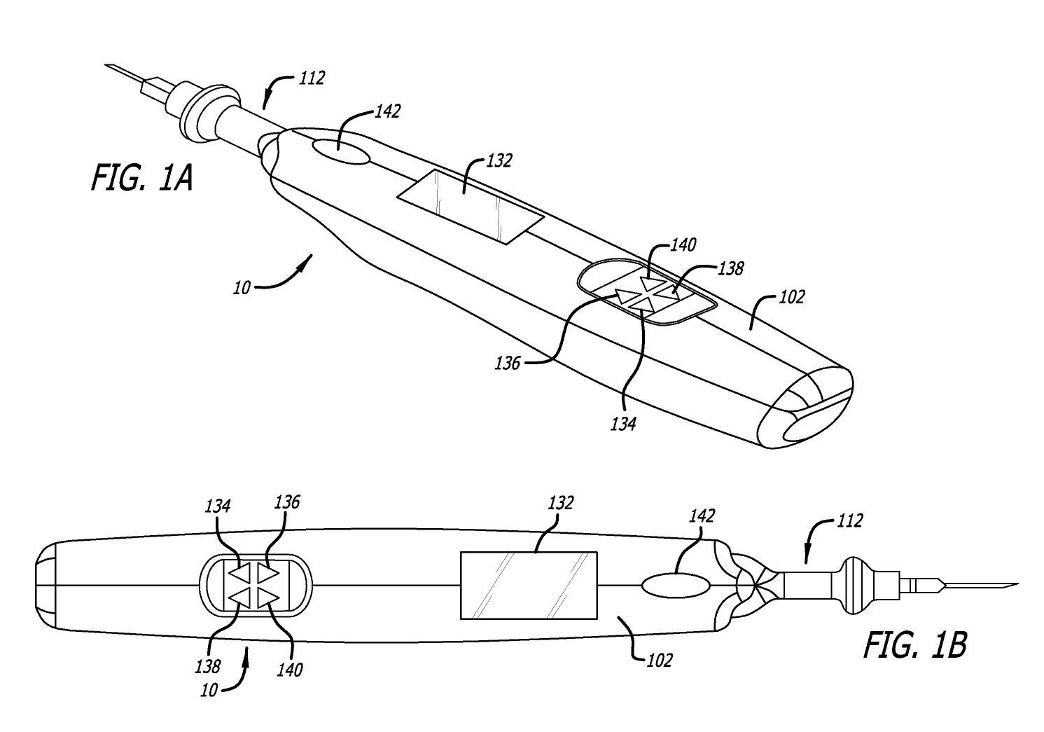

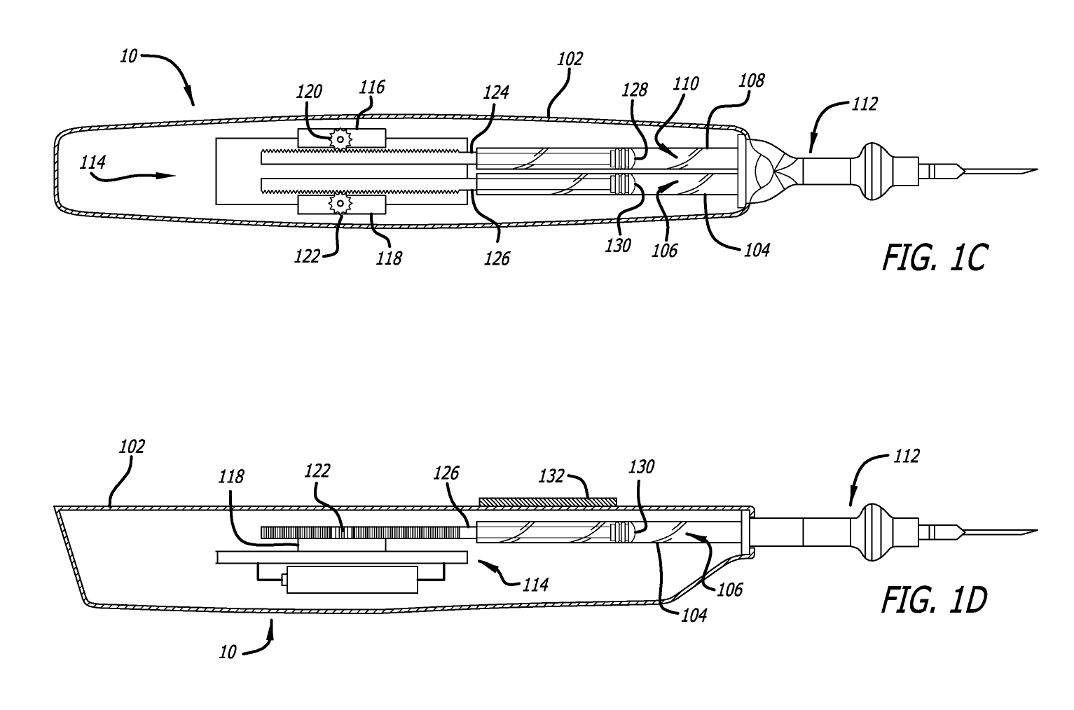

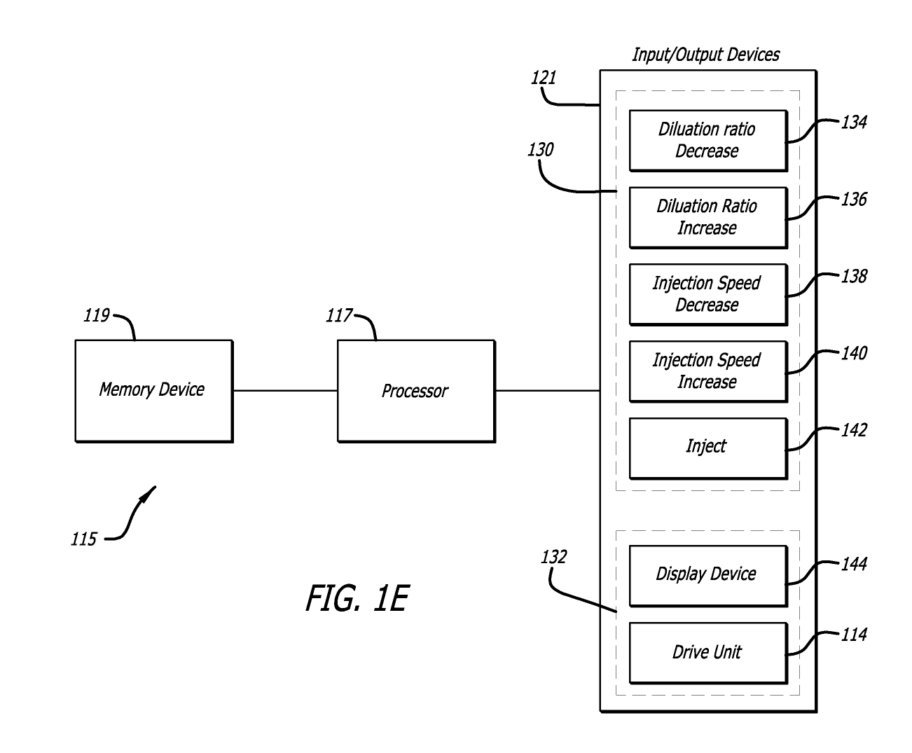

Referring now to FIGS. 1A through 1E, in one embodiment, injection device 10 includes: (a) housing or body 102; (b) first cartridge 104 defining a first chamber 106 which is configured to contain a first injectable fluid; (c) second cartridge 108 defining a second chamber 110 which is configured to contain a second injectable fluid; (d) mixing unit 112 configured to mix the first injectable fluid and the second injectable fluid; (e) drive unit 114; (f) control system 115 having: (i) processor 117; (ii) memory device 119; and (iii) input/output devices 121.

Housing 102 may have a grippable housing, which may be made of any suitable material, e.g., metals, thermoplastics, thermoplastic elastomers (TPEs), silicones, glass, etc., or any combination of materials. Housing 102 may be shaped to comfortably accommodate a user's hand. A portion of housing 102 designed to be gripped may be textured to provide a secure grip, or may be covered in a layer of material designed to provide a secure grip.

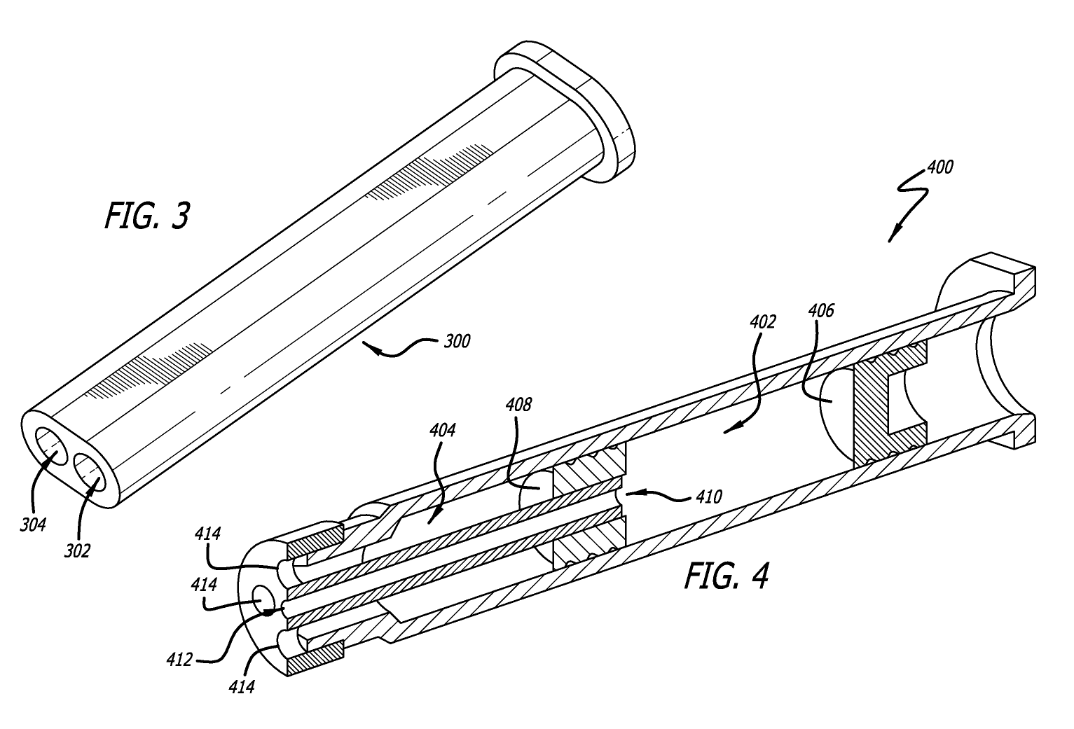

As illustrated in FIG. 10, in this example, first cartridge 104 is separate from second cartridge 108. In an alternative example, injection device 10 includes two cartridges combined into a single component. For example, as illustrated in the FIG. 3, cartridge 300 includes two cartridges combined into a single component. Cartridge 300 defines first chamber 302 for containing the first injectable fluid and second chamber 304 for containing the second injectable fluid. In this example, chambers 302 and 304 are configured to receive different plungers which facilitate a part of the extrusion process. The geometry of these cartridges are not fixed, as long as they can hold a minimum of 1 mL of fluid.

In one alternative example, injection device 10 includes a single cartridge which defines a plurality of chambers which contain the fluids. Referring to FIG. 4, single cartridge 400 defines: (a) first chamber 402 configured to contain the first injectable fluid; and (b) second chamber 404 configured to contain the second injectable fluid. In this example, single cartridge 400 includes first plunger head 406 and second plunger head 408. Single cartridge 400 forms center channel 410. In operation, when first plunger head 406 is pushed forward, the first injectable fluid is caused to flow from first chamber 402 through center channel 410 and out of the end 412 of center channel 410. In response to second plunger head 408 being pushed forward, the second injectable fluid is caused to flow from second chamber 404 through output channels 414 of single cartridge 400.

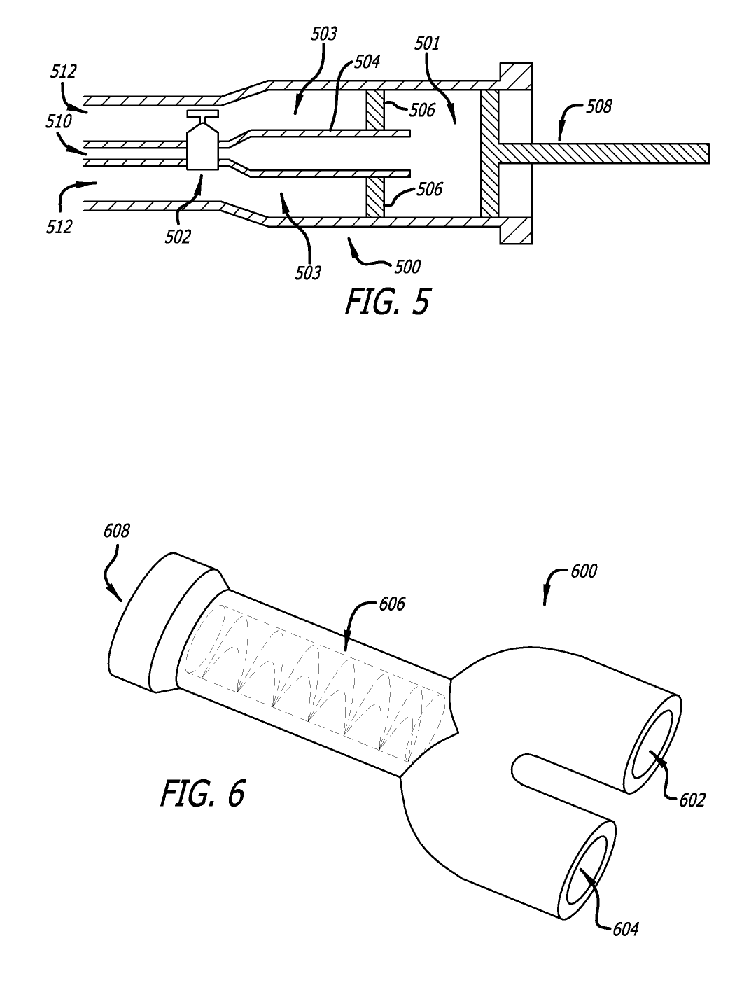

In one embodiment, where injector device 10 includes a single cartridge, the single cartridge is operatively connected to a flow/pressure regulator. For example, as illustrated in FIG. 5, single cartridge 500 is operatively connected to flow/pressure regulator 502. Single cartridge 500 defines: (a) first chamber 501 configured to contain the first injectable fluid; and (b) second chamber 503 configured to contain the second injectable fluid. In this example, single cartridge 500 has center stem 504. Single cartridge 500 includes first plunger 506 which has a hole portion and forms a seal around center stem 504. In operation, when second plunger 506 is pushed forward, the first injectable fluid is caused to flow through center stem 504 and out of the end 510 of center stem 504. In response to first plunger 508 being pushed forward, the second injectable fluid is caused to flow through output channel 512 of single cartridge 500. In this example, when flow/pressure regulator 502 is in a closed position, only the second injectable fluid from the front half is allowed to flow out from single cartridge 500. As flow/pressure regulator 502 is opened, a greater percentage of the first injectable fluid from the back half will be driven out. In this example, the dilution ratio of the mixed fluid is determined based on the amount of fluid flow through flow/pressure regulator 502. In this example, using encoders (not shown), injection device 10 monitors the amount of fluid which has passed. Injection device 10 determines the location of first plunger 508 using any one of the described methods herein. Injection device 10 determines the location of second plunger 506 by monitoring the flow out of any one of channels 510 and 512, or by using a linear encoder (not shown) which determines the linear position of second plunger 506. In this example, flow/pressure regulator 502 is positioned in line with center stem 504. In an alternate example, flow/pressure regulator 502 is positioned in line with outer channel 512. It should be appreciated that the energy source which drives the plunger can be any suitable energy source such as any of the energy sources described herein (e.g., gear motors, pressure source, nitinol actuators, etc.).

In one embodiment, the injection device attaches to and operates a standard needle and cartridge combination. That is, in this example, the injection device does not include any cartridges. Rather, the injection device is configured to receive and operate with the cartridges. In one embodiment, the injection device is attached the cartridge using a luer slip or luer lock attachment. In one embodiment, the cartridges include a protruding or snap feature used to lock the cartridges into the injection device when it is fully inserted. In one embodiment, the inner body of the injection device includes the protruding or snap feature. In one embodiment, the cartridges include a ring which seals the cartridges into cartridge slots of the injection device.

The injection device may also include a cartridge retention and ejection mechanism. This mechanism may facilitate loading of, e.g., pre-filled, disposable cartridges. The mechanism may also provide for the rotation of cartridges.

In one embodiment, the injection device houses chambers itself to contain fluids to be injected. In this example, a needle is attached directly to the injection device. In one embodiment, the injection device includes a cartridge housing in which the cartridge(s) may be secured. In one embodiment, the cartridge housing is substantially in the form of a tube. The cartridge housing may be designed to hold a disposable, pre-filled cartridge. The cartridge housing may be all or partially transparent, allowing a user to view the cartridge during operation. For example, the cartridge housing may provide a user with a view of both a cartridge in the housing and also a cartridge plunger which may extrude fluid from the cartridge when the injection device is in operation.

In one embodiment, the cartridge(s) is made of cyclic olefin copolymer (COC). Any other suitable materials may be utilized.

In one embodiment, each cartridge is filled using different dedicated filling tips. Once filled, a sealing tip may be employed to prevent mixing of the injectable fluids while in storage.

In one embodiment, the cartridge includes a needle. In one embodiment, the cartridge is configured to be coupled to a needle. In one embodiment, using a luer tip, the at least one cartridge is coupled to a needle. The needle itself may have any suitable gauge, for example, a gauge between about 10 and about 33. In one embodiment, the needle is a 30G×¾″ needle.

In one embodiment, after a desired amount of fluid has been injected into the patient, the user of the injection device may remove and discard the used cartridge(s) along with the needle.

It should be appreciated that, in different examples, the injection device 10 is configured to include or attach to any suitable cartridge.

In one embodiment, the injectable fluids (e.g., the first injectable fluid or the second injectable fluid) include at least one biocompatible material. These biocompatible materials include, but are not limited to, dermal fillers, hyaluronic acid-based dermal fillers (e.g., Juvederm™ Ultra and Juvederm™ Ultra Plus (Allergan, Irvine, Calif.)), hydrogels (i.e., superabsorbent natural or synthetic polymers), organogels, xerogels, encapsulated and/or cross-linked biomaterials, silicones, glycosaminoglycans (e.g., chondroitin sulfate, dermatin sulfate, dermatin, dermatin sulfate, heparin sulfate, hyaluronic acid, o-sulfated hyaluronic acid), polysaccharides (e.g., chitosan, starch, glycogen, cellulose), collagen, elastin, local anesthetics (e.g., Benzocaine, Chloroprocaine, Cyclomethycaine, Dimethocaine/Larocaine, Propoxycaine, Procaine/Novocaine, Proparacaine, Tetracaine/Amethocaine, Amino amides, Articaine, Bupivacaine, Carticaine, Cinchocaine/Dibucaine, Etidocaine, Levobupivacaine, Lidocaine/Lignocaine, Mepivacaine, Piperocaine, Prilocalne, Ropivacaine, Trimecaine), drugs, bioactive agents, antioxidants, enzyme inhibitors (e.g., anti-hyaluronidase), vitamins, minerals, water, saline, light curable or light activated materials, vaccines, and pH curable or pH activated materials. Other biocompatible materials not mentioned above are also considered within the scope of the present description.

In one embodiment, the second injectable fluid includes a bioactive agent which facilities delivery of the first injectable fluid injection (e.g., to reduce extrusion force and/or viscosity). Additional bioactive agents may include anti-proliferatives including, but not limited to, macrolide antibiotics including FKBP-12 binding compounds, estrogens, chaperone inhibitors, protease inhibitors, protein-tyrosine kinase inhibitors, leptomycin B, peroxisome proliferator-activated receptor gamma ligands (PPARy), hypothemycin, nitric oxide, bisphosphonates, epidermal growth factor inhibitors, antibodies, proteasome inhibitors, antibiotics, anti-inflammatories, anti-sense nucleotides and transforming nucleic acids. Drugs can also refer to bioactive agents including anti-proliferative compounds, cytostatic compounds, toxic compounds, anti-inflammatory compounds, anti-fungal agents, steroids, chemotherapeutic agents, analgesics, antibiotics, protease inhibitors, statins, nucleic acids, polypeptides, growth factors and delivery vectors including recombinant micro-organisms, liposomes, and the like. Combinations of additional bioactive agents are also within the scope of the present description.

Other injectable fluids (e.g., the first injectable fluid or the second injectable fluid) may include toxins such as botulinum toxins. The botulinum toxin can be selected from the group consisting of botulinum toxin types A, B, C₁, D, E, F and G, a pure or purified (i.e., about 150 kD) botulinum toxin, as well as a native or recombinant botulinum toxin. The material can comprise between about 1 unit to about 20,000 units of the botulinum toxin or a therapeutically effective amount, and the composition can comprise an amount of botulinum toxin sufficient to achieve a therapeutic effect lasting between 1 month and 5 years. The botulinum toxin can be reconstituted within the device as described elsewhere herein or before the cartridge is placed in the device. The botulinum toxin can be reconstituted with sterile 0.9% sodium chloride (saline).

In one embodiment, the dilution ratio is 1 to 100 units of botulinum toxin per 0.1 mL of saline. More preferably, in one embodiment, the dilution ratio is 1 to 50 units per 0.1 mL of saline, or 1 to 10 units per 0.1 mL of saline. In one embodiment, 4 units per 0.1 mL of saline is used. The dilution ratio will be highly dependent on the type of botulinum toxin used or combination of botulinum toxins used.

In one embodiment, mixing unit 112 is configured to mix injectable fluids by directing the injectable fluids into a spiral mixing path. For example, as illustrated in FIG. 6, mixing unit 600 defines: (a) first input channel 602; (b) second input channel 604; (c) spiral mixing path 606; and (d) output channel 608. In operation, as injectable fluids simultaneously move, from input channels 602 and 604, through spiral mixing path 606, the injectable fluids can mix together to produce the injectable mixed fluid. In one embodiment, the mixing unit 600 is rotationally symmetric such that each piece can be mated to an identical piece.

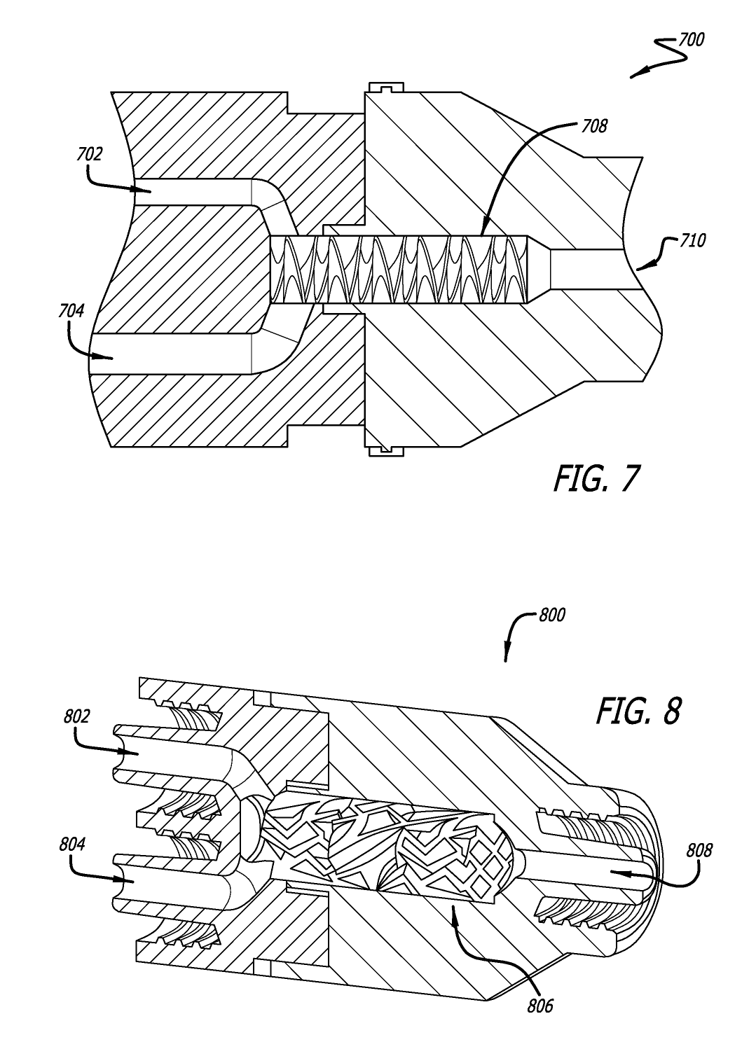

In one embodiment, mixing unit 112 is configured to mix fluids by directing the injectable fluids into a helical path. For example, as illustrated in FIG. 7, mixing unit 700 defines: (a) first input channel 702; (b) second input channel 704; (c) helical mixing path 708; and output channel 710. In operation, as the injectable fluids simultaneously move, from input channels 702 and 704, through helical mixing path 708, the injectable fluids mix together to produce the injectable mixed fluid. Each mixer segment piece provides a single helical revolution in the opposite direction (i.e., clockwise vs. counterclockwise). The helical shape causes a significant amount of turbulence by causing the injectable fluids to change direction.

In one embodiment, mixing unit 112 includes corrugated sections which are configured to mix the injectable fluids. For example, as illustrated in FIG. 8, mixing unit 800 defines: (a) first input channel 802; (b) second input channel 804; (c) corrugated sections 806; and (d) output channel 808. In operation, as injectable fluids simultaneously move, from input channels 802 and 804, through corrugated sections 806, the injectable fluids mix together to produce an injectable mixed fluid. In this example, the corrugated sections are offset by 90° angles to each other. In each section, the corrugations run at 45° angles to the unobstructed injectable fluid path. The layers of corrugation with each section like 90° out of phase with each other.

Each of the mixing units described above have been static. It should be appreciated that in other examples, the mixing unit may be dynamic. It should also be appreciated that in different examples, the injection device may include any suitable mixing unit, including any of the mixing units described herein.

As illustrated in FIGS. 10 and 1D, in this example, drive unit 114 includes first gear motor 116 and second gear motor 118. First gear motor 116 is operatively connected to first gear 120, and second gear motor 118 is operatively connected to second gear 122. In this example, drive unit 114 also includes: (a) first rack 124 which is operatively engaged with the first gear 116 and first plunger 128; and (b) second rack 126 which is operatively engaged with the second gear 122 and second plunger 130. Drive unit 114 illustrated in FIGS. 10 and 1D may provide an effectively infinite number of dilution ratios and injection speeds by independently setting the speed of one gear motor relative to another gear motor.

In operation, in this example, drive unit 114 drives the linear motion of plungers 128 and 129 which causes the fluids to be extruded. More specifically, first gear motor 116 causes first gear 120 to turn, thereby driving the linear motion of first rack 124. First rack 124 engages first plunger 128, thereby causing the first injectable fluid to flow from the first chamber to mixing unit 112. Second gear motor 118 causes second gear 122 to turn, thereby driving the linear motion of second rack 216. Second rack 126 engages second plunger 130, thereby causing the second injectable fluid to flow from the second chamber to mixing unit 112.

In one embodiment, the rotational output of the motors drives the linear motion of the racks through worm gears. In another example, the rotational output of the motors drive the linear motion of the racks through concentric gearing of an internally threaded gear to a threaded rack.

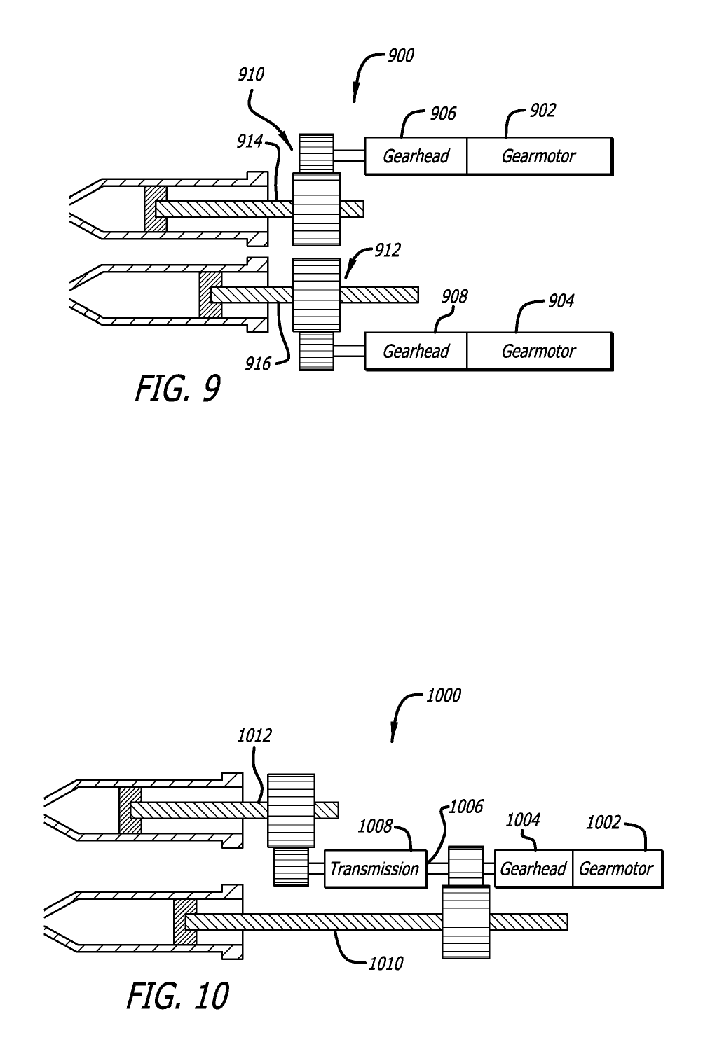

FIG. 9 illustrates a schematic diagram of one embodiment of the drive unit, illustrating the drive unit having independent dual gear motors. Drive unit 900 includes: (a) gear motors 902 and 904; (b) gearheads 906 and 908; (c) gear assemblies 910 and 912; and (d) plungers 914 and 916. In operation, gear motors 902 and 904 are driven through gearheads 906 and 908 to achieve necessary speed reduction and force multiplication. The rotation output from gear motor 902 drives the linear motion of plunger 914 through gear assembly 914. Similarly, the rotation output from gear motor 904 drives the linear motion of plunger 916 through gear assembly 912. Described in more detail below with reference to FIGS. 10 to 13, in different embodiments, the drive unit may include a single gear motor and a transmission, a pressure driven system, a hydraulically driven system, or a nitinol driven system.

It should be appreciated that any of the motors discussed herein may be any suitable electric motor capable of supplying the necessary force. In one embodiment, the motors are operatively connected to the plungers via certain of the drive units discussed herein. In some examples, the drive units function to transfer the rotational motion of the motors into the linear motion of the plunger.

In one embodiment, the injection device includes a control system. In one embodiment, the control system may include at least one processor, at least one memory device operatively connected to the at least one processor, at least one input device operatively connected to the at least one processor, and at least one output device operatively connected to the at least one processor.

The at least one processor may be any suitable processor unit of a kind normally used in such devices. In one embodiment, the control system includes one or more digital processors, such as a digital microprocessor or a micro-controller based platform. In one embodiment, the control system includes one or more analog control units such as a suitable integrated circuit or one or more application-specific integrated circuits (ASIC's). In one embodiment, the control system is in communication with, or operable to access or exchange signals with the at least one memory device. In this example, the memory device stores program code or instructions, executable by the processor(s), to control the injection device. In one embodiment, such memory device includes: (a) RAM (MRAM); (b) ferroelectric RAM (FeRAM); (c) read only memory (ROM); (d) flash memory; (e) EEPROM (electrically erasable programmable read only memory); or a suitable combination of such memory devices. It should be appreciated that any other suitable magnetic, optical, or semiconductor memory may operate in conjunction with, or as part of, the injection device.

In one embodiment, the output devices include at least one display device. In one embodiment, the display device includes an LCD screen which is located on a front of the injection device, and allows a user to interact with the system. In one embodiment, the LCD screen displays a dot matrix pattern. In one embodiment, the display device includes LED technology. In one embodiment, the injection device causes an LED display device to display proprietary artwork. In one embodiment, the display device includes electroluminescent panels. In one embodiment, the display device includes an interface. Using the interface, the user may control the operation of the device.

The injection device may be configured to cause the display device to display at least one of, for each fluid contained: (i) the volume that has been injected; (ii) the volume remaining; (iii) the starting volume; and (iv) the speed or injection rate. The display device may also display at least one of: (a) the total volume of fluid that has been extruded or injected; (b) the speed or rate of injection of the mixed fluid; (c) the dilution ratio of the fluid being injected. In addition, other information may be displayed to facilitate different functions. For instance, the display device may also display configuration screens, summary information, error indicators in the case of a malfunction, and/or battery power information.

In one embodiment, the input devices include an inject button. The inject button may be located on injection device 10 in a position which is conveniently accessible by a user's fingers or thumb during injection. The inject button may start and stop the injection process. In one embodiment, the user may press and hold the inject button to begin the injection, and may release the inject button to stop the injection. In other examples, the injection process may work in other ways. For instance, the user may press the inject button once to begin the injection and a second time to stop the injection. In other embodiments, the injection process starts based on a user pressing switch or some other actuator.

In one embodiment, control system 115 includes at least one input device (e.g., a keypad, a button, a dial or a switch) which enables a user to control the overall speed or rate or volume of the extrusion. In one embodiment, control system 115 includes at least input device (e.g., a button, dial or switch) which enables a user to control the overall speed or rate or volume of the injection by enabling the user to independently control the speed or rate or volume of the injection of each injectable fluid.

In one embodiment, the injection device is configured to extrude fluid at a plurality of predetermined selectable speeds. As described in more detail below, in one embodiment, the injection device is configured to extrude fluid at the following four different selectable speeds: very low, low, medium and high. In one embodiment, the injection device is configured to extrude fluid at a dynamic speed which enables extrusion of each of the four different speeds based on the amount of pressure exerted on the inject button. Lighter pressure on the inject button will correspond to a lower injection speed and a higher pressure will correspond to a higher injection speed. The approximate corresponding flow rates are shown in Table 1.

These flow rates were determined based on evaluation physician's typical extrusion rates.

TABLE 1
Exemplary Injection Rates

| Speed Setting | Injection Rate (+/−0.20 mL/minute)* |
|---|---|
| Very Low | 0.30 |
| Low | 0.60 |
| Medium | 0.90 |
| High | 1.20 |
| Dynamic | 0.30-1.20 |

*APPROXIMATE INJECTION RATE

In one embodiment, the input devices include at least one encoder. Using at least one encoder, the injection device determines the position of the plungers. For example, the injection device illustrated in FIGS. 1A through 1E uses a first encoder (not shown) to determine the position of the first plunger, and uses a second encoder (not shown) to determine the position of the second plunger. In this example, these encoders are preferably located on the gear motors. In one embodiment, using the at least one encoder, the injection device determines and displays volume information of each contained fluid and/or the total volume extruded/injected.

In one embodiment, the encoder is rotational encoder connected to a motor. In this example, the rotational encoder is configured to sense the rotation of the motor. For example, the motor may rotate a portion of the rotational encoder.

In different examples, other portions of the injection device may be encoded. For example, in one embodiment, the injection device includes a separate linear encoder for each of the plungers.

Referring to FIG. 1E, control system 115 includes at least one processor 117; at least one memory device 119 operatively connected to processor 117; input devices 130 operatively coupled to processor 117; and output devices 132 operatively coupled to processor 117. In this example, as illustrated in FIGS. 1A, 1B and 1E, input devices 130 include: (a) dilution ratio decrease button 134; (b) dilution increase button 136; (c) injection speed decrease button 138; (d) injection speed increase button 140; and (e) inject button 142. Output devices 132 include: (a) display device 144; and (b) drive unit 114. Control system 115 may be a portion of a control system for the injection device (not shown).

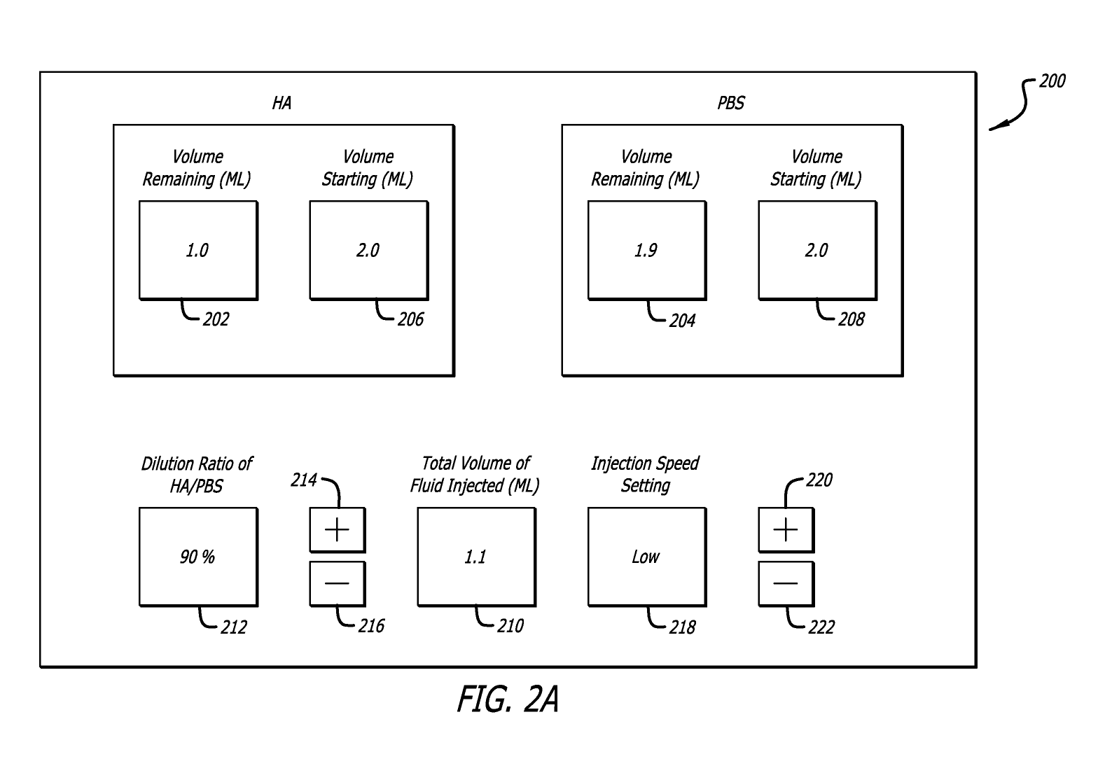

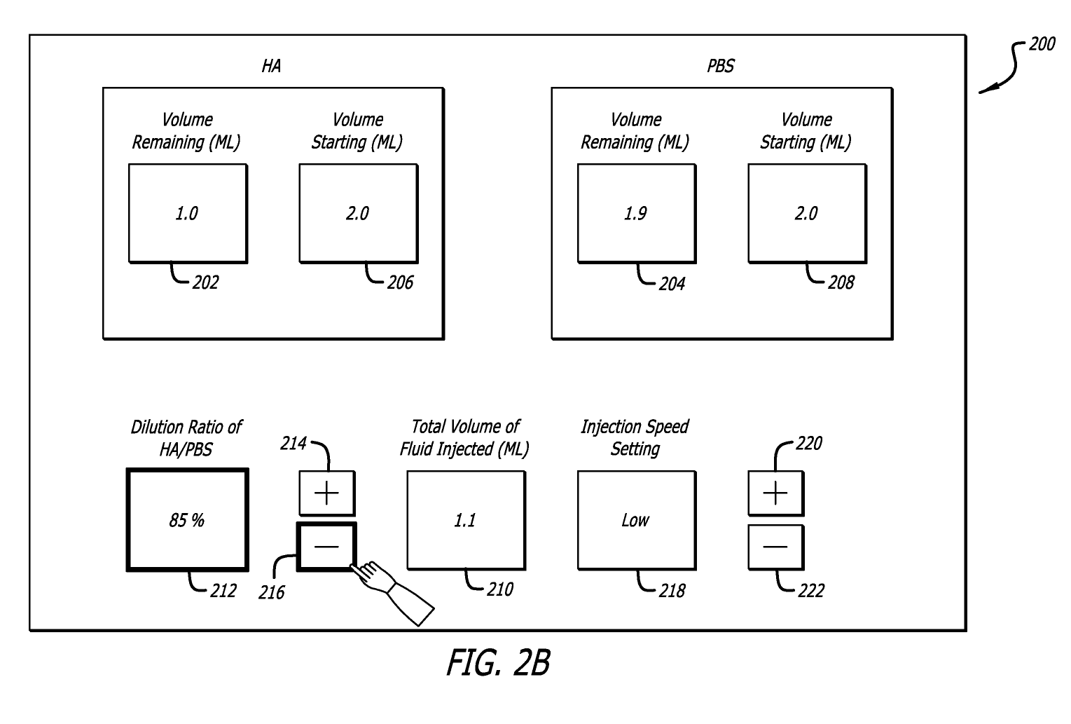

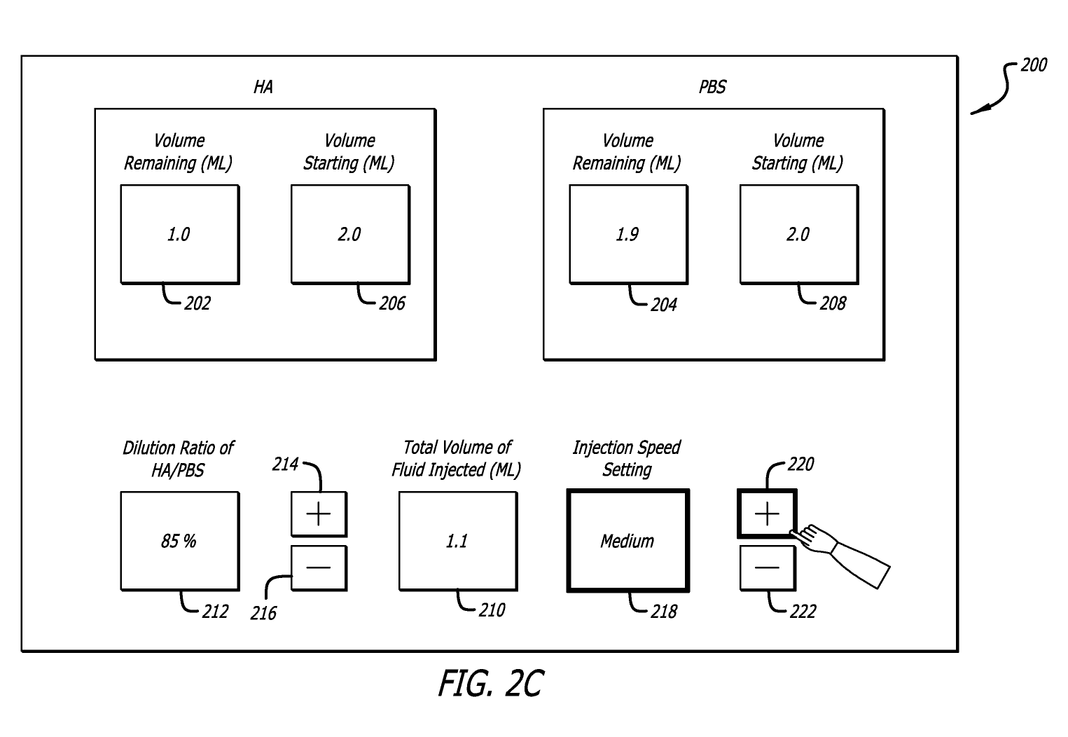

Referring to FIGS. 2A to 2C, this example generally shows an example illustrating: (a) the selection of a dilution ratio of injectable fluids to produce a mixed fluid; and (b) for the mixed fluid, the selection of an injection or extrusion speed. In this example, the first injectable fluid is HA and the second injectable fluid is PBS. In this example, the injection device provides a mixed fluid made up of HA and PBS based on the selected dilution ratio, and extrudes the injectable mixed fluid based on the selected injection speed. It should be understood that although in this example, the fluids include HA and PBS, in different examples the fluids may include any suitable fluid which is desired to be diluted or mixed.

FIG. 2A illustrates a point in time in which 1.1 mL of injectable fluid had previously been extruded from the injection device.

In this example, display device 200 displays first volume remaining meter 202 for the HA, and second volume remaining meter 204 for the PBS. First volume remaining meter 204 displays the amount or volume of HA remaining. At the point in time illustrated in FIG. 2A, first volume remaining meter 202 indicates that 1.3 mL of HA remain. Second volume remaining meter 204 displays the amount or volume of PBS remaining. At the point in time illustrated in FIG. 2A, second volume remaining meter 204 indicates that 1.9 mL of HA remain.

Display device 200 also displays first volume starting meter 206 for the HA, and second volume starting meter 208 for the PBS. First volume starting meter 206 displays the amount or volume of HA which the injection device started with before the extrusion process. In this example, first volume starting meter 206 indicates that, before the extrusion process, the injection device included 2.0 mL of HA. Second volume starting meter 208 displays the amount or volume of PBS which the injection device started with before the extrusion process. In this example, second volume starting meter 208 indicates that, before the extrusion process, the injection device included 2.0 mL of PBS.

Display device 200 also displays total volume of fluid injected or extruded meter 210. Total volume of fluid injected meter 210 displays the total amount or volume of fluid which has been injected or extruded. At the points in time illustrated in FIGS. 2A to 2C, the total volume of fluid injected meter 210 indicates that 1.1 mL of total fluid had previously been injected.

Display device 200 also displays dilution ratio meter 212. In this example, dilution meter 212 displays the ratio of HA to PBS. At the point in time illustrated in FIG. 2A, dilution ratio meter 212 indicates a 90% ratio (i.e., 90% HA and 10% PBS).

Display device 200 also displays dilution ratio increase button 214 and dilution ratio decrease button 216. In this example, the user is enabled to control the specific dilution ratio by selecting dilution ratio increase button 214 and dilution ratio decrease button 216. For example, as illustrated in FIG. 2B, the user pushes dilution ratio decrease button 216. In this example, injection device 10 displays an indication (i.e., the highlighted borders) to the user which indicates that dilution ratio decrease button 216 has been selected. In FIG. 2B, based on the selection, dilution meter 212 indicates a dilution ratio of 85% (i.e., 85% HA and 15% PBS). In this example, the selection of dilution ratio decrease button 216 causes injection device 10 to control the extrusion of the mixed fluid such that any extruded mixed fluid has a dilution ratio of 85% (i.e., 85% HA and 15% PBS).

Display device 200 also displays injection speed setting meter 218. In this example, injection speed setting meter 218 displays the current injection speed setting of the injection device. At the point in time illustrated in FIG. 2A, injection speed setting meter 218 indicates a Low speed is set for the injection device. In this example, a Low speed setting corresponds to a injection rate of about 0.60 mL per minute.

Display device 200 also displays injection speed increase button 220 and injection speed decrease button 222. In this example, the user is enabled to control the specific injection rate speed by selecting injection speed increase button 220 and injection speed decrease button 222. For example, as illustrated in FIG. 2C, the user pushes injection speed increase button 220. In this example, injection device 10 displays an indication (i.e., the highlighted borders) to the user which indicates that injection speed increase button 220 has been selected. In FIG. 2C, based on the selection, injection speed setting meter 218 indicates a Medium speed is set for an injection rate. In this example, a Medium speed setting corresponds to an injection rate of about 0.90 mL per minute.

In one embodiment, the injector device determines the ratio of the first fluid and the second fluid based on the selected injection speeds of the first fluid and the second fluid. That is, in this example, the injection device enables a user to select a first injection rate for the first fluid and a second injection rate for the second fluid. After the injection rates have been selected or set, in response to the user selecting the inject button, the injection device causes each of the injectable fluids to extrude the injection device based on their selected injection rates.

It should be understood that, in one example, the user is enabled to cause the injection device to select a dilution ratio of 100% (e.g., 100% HA and 0% PBS).

In one embodiment, drive unit 114 includes a single gear motor and a transmission. For example, drive unit 1000 illustrated in FIG. 10 includes: (a) single gear motor 1002; (b) gearhead 1004; (c) output shaft 1006; (d) transmission 1008 having: (i) input configured to receive the output shaft 1006; and (ii) gear ratios; (e) first plunger 1010; and (e) second plunger 1012. In this example, output shaft 1006 of single gear motor 1002 is connected to the input 1006 of transmission 1008 which in turn drives second plunger 1012. In this example, the transmission's gear ratios are selected such that each gear will deliver a desired dilution ratio. In one embodiment, drive unit 1000 includes a separate energy source (not shown) to switch gears in transmission 1008. In different embodiments, the gears are switched in transmission 1008 using an additional motor, a user operated switch, and/or a nitinol actuator. In one embodiment, using a single encoder (not shown) on the single gear motor 1002, the injection device determines the positions of first plunger 1010 and second plunger 1012 based on the amount of time the transmission was engaged in each gear.

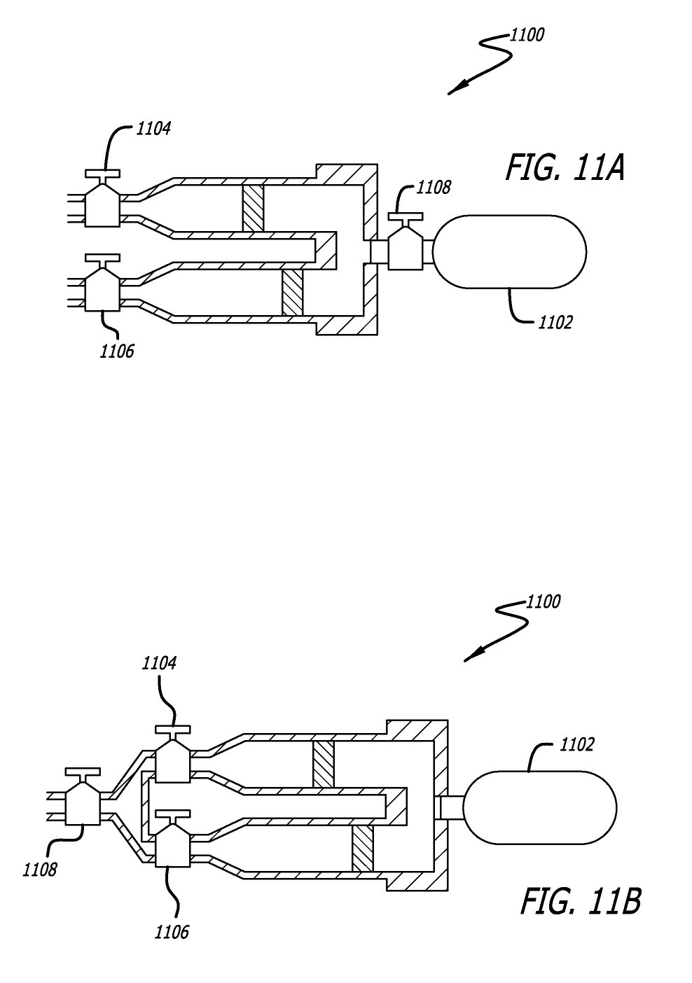

In one embodiment, drive unit 114 includes a pressure driven system which includes a pressure source (e.g., a CO2 cartridge) used to drive each plunger forward. In this embodiment, the dilution ratio is determined by regulating the flow of the fluid from each cartridge. In one embodiment, the injection device enables a user to regulate each cartridge by manually control the individual flow out of the cartridges using pressure/flow regulators or variable orifice valves. In one embodiment, the injection device electronically controls the individual flow out of the cartridges using pressure/flow regulators. Referring to FIG. 11A, in this example, drive unit 1100 includes: (a) pressure source 1102; (b) first regulator 1104; (c) second regulator 1106; and (d) third regulator 1108. In this example, the net flow through the injection device 10 is controlled by the third regulator 1108 being positioned at the inlet to the cartridges. In another example, as illustrated in FIG. 11B, the net flow through injection device 10 is controlled by third regulator 1108 being positioned at a location after the fluids have mixed. It should be appreciated that, where the drive unit of the injection device includes a pressure driven system, many pressure/flow regulator combinations may be used to control injection rate and dilution ratio. In this example, the injection device may determine the amount of fluid which has been injected/extruded using encoders which indicate the positions of the plungers,

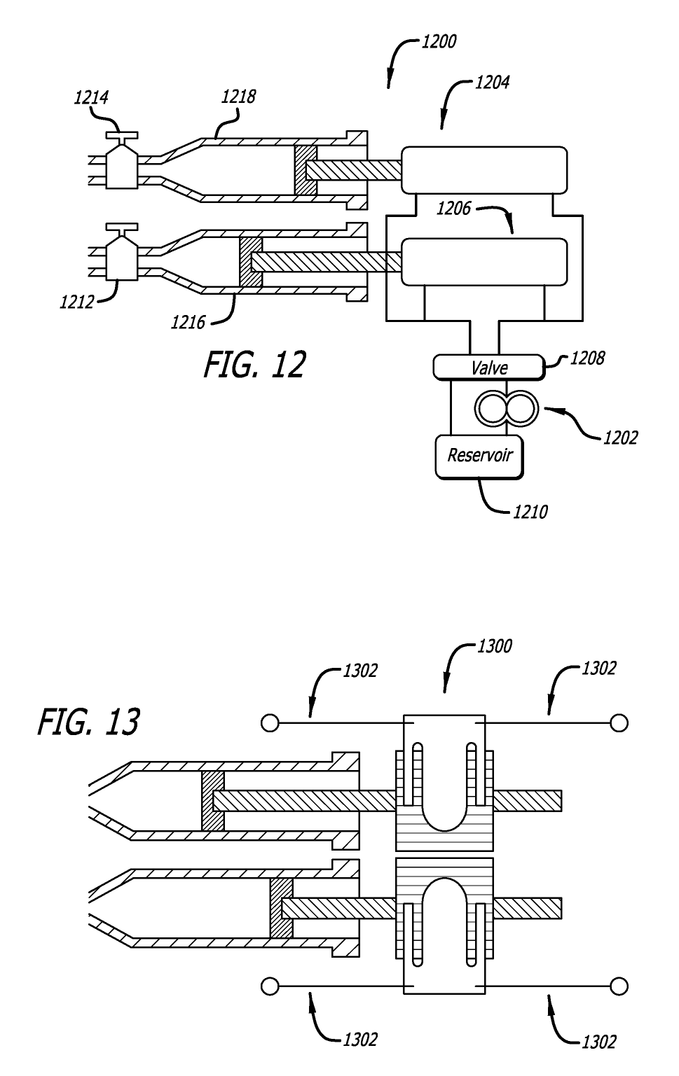

In one embodiment, drive unit 114 includes a hydraulically driven system. For example, as illustrated in FIG. 12, drive unit 1200 includes: (a) pump 1202; (b) first hydraulic piston 1204; (c) second hydraulic piston 1206; (d) valve 1208; (e) reservoir 1210; (f) first regulator 1212; and (g) second regulator 1214. In this example, injection device 10 uses pump 1202 to activate hydraulic pistons 1204 and 1206. The hydraulic pistons force the plungers forward which drive fluid out of cartridges 1216 and 1218. In this example, injection device 10 uses regulators 1212 and 1214 to control the flow of the fluids out of cartridges 1212 and 1214. In one embodiment, injection device 10 enables a user to manually control the individual flow out of the cartridges using the pressure/flow regulators. In one embodiment, the injection device electronically controls the individual flow out of the cartridges using pressure/flow regulators. In one embodiment, the dilution ratio is determined by the relative regulation of each cartridge. In one embodiment, after the completion of an injection, valve 1208 is toggled. This allows pump 1202 to drive hydraulic fluid into the front of the pistons, retracting the plungers quickly.

In one embodiment, drive unit 114 includes a nitinol drive system. For example, as illustrated in FIG. 13, drive unit 1300 includes shape memory actuators 1302. In this example, shape memory actuators 1302 are wire-shaped and are made of nitinol or some other material that changes shape or size. Although in this example, four wires are illustrated, it should be appreciated that in different examples, the injection device may include any suitable amount of wire-shaped memory actuators. When an electrical current is applied to shape memory actuators 1302, the shape memory actuators 1302 shorten a specific amount. More specifically, the electric current causes the wires 1302 to heat, which in turn triggers its length transformation. Each pair of opposing wires turns off and on in sequence, causing a ratcheting member to toggle back and forth. It should be appreciated that any number of wires may be used in parallel to increase force or to increase plunger speed. In one embodiment, the injection device determines the location of the plungers by counting the number of actuations of wires 1302 and correlating the count with plunger movement.

In different examples, injection device 10 may be ergonomically designed to facilitate injection for a wide variety of hand shapes, sizes and gripping positions. Advantageously, the injector device may be easy to manipulate and grip. In alternative embodiments, heavier components of the device are housed in the different positions of the injection device.

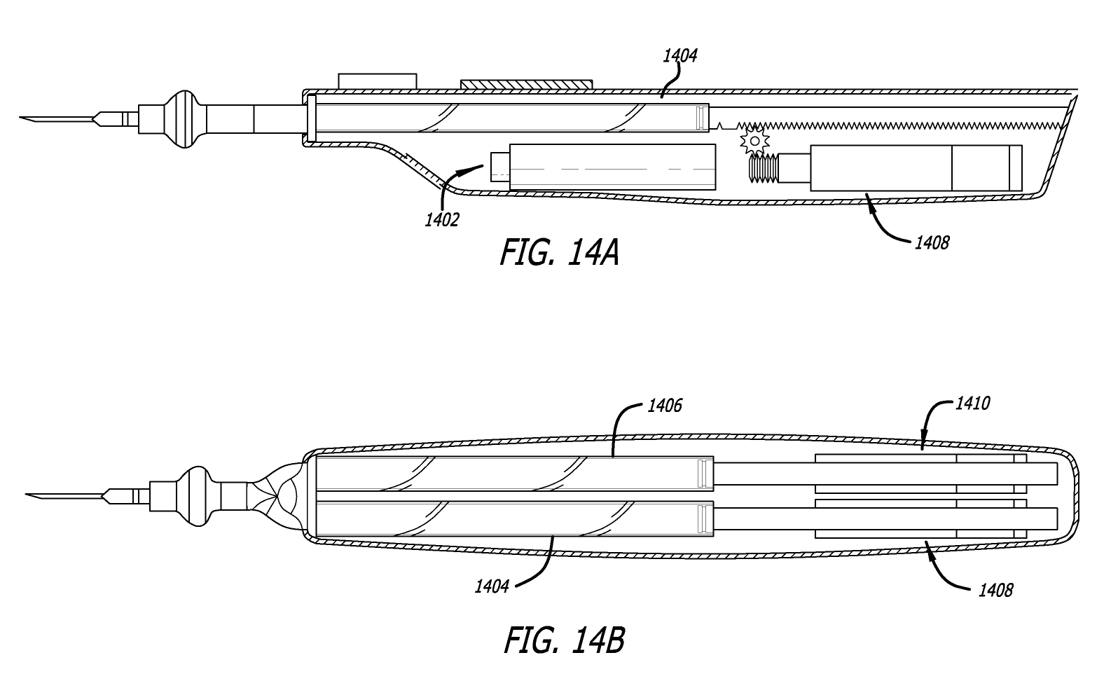

Discussed in more detail below, FIGS. 14A, 14B, 15, 16, 17, 18, 19, 20A and 20B illustrate different component configurations which may provide a more balanced device (e.g., weight balance, ergonomically balanced, etc.) and facilitate injection for a wide variety of hand shapes, sizes and gripping positions.

In one embodiment, the components of the injection device 10 are configured such that the weight of injection device 10 is effectively balanced by positioning batteries and cartridges in the front section of the injection device 10, and motors in the rear section of injection device 10. For example, as illustrated in FIGS. 14A and 14B, first battery 1402 is positioned under first cartridge 1404, and second battery (not shown) is positioned under second cartridge 1406. Motors 1408 and 1410 of injection device 1400 are positioned in the rear section of injection device 1400. In this example, the cross section of the injection device is fairly consistent along its length.

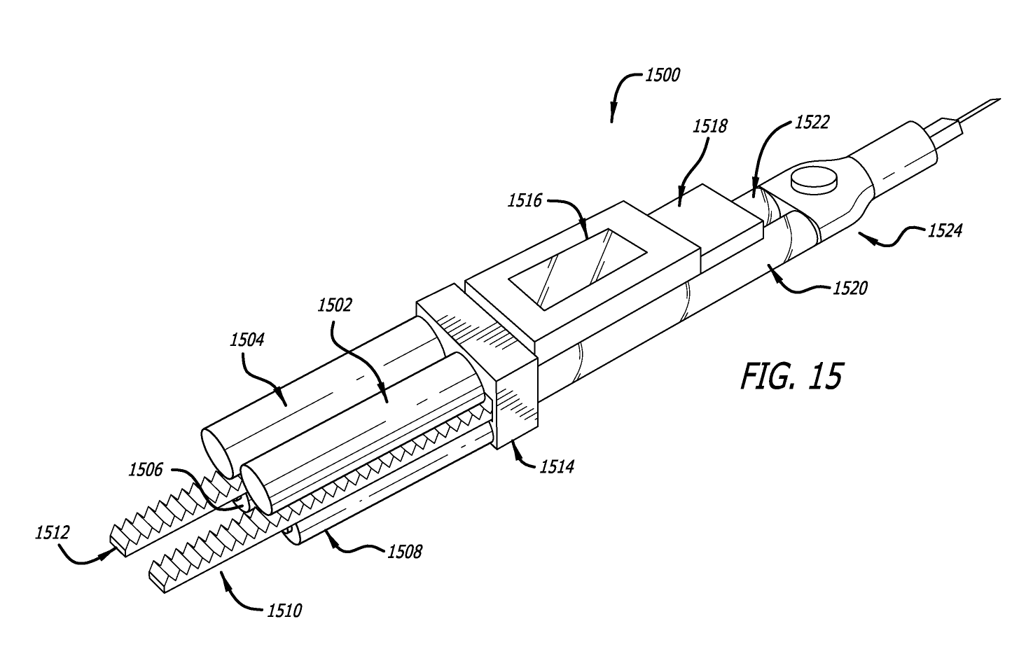

In one embodiment, the components of injection device 10 are configured such that batteries and motors of the injection device are positioned at the rear section of injection device 10. For example, as illustrated in FIG. 15, injection device 1500 includes: (a) first motor 1502; (b) second motor 1504; (c) first battery 1506; (d) second battery 1508; (e) first rack 1510; (f) second rack 1512; (g) gearbox 1514: (h) display device 1516 which functions as a user interface; (i) circuit board 1518; (j) first cartridge 1520; (k) second cartridge 1522; and (l) mixing unit 1524. In this example, display device 1516 is positioned approximately halfway along injection device 1500 length, minimizing the eye travel between the treatment site and the display device 1516. Batteries 1506 and 1508 and motors 1502 and 1504 are located at the rear of injection device 1500. In this example, the cross section of injection device 1500 is fairly consistent along its length.

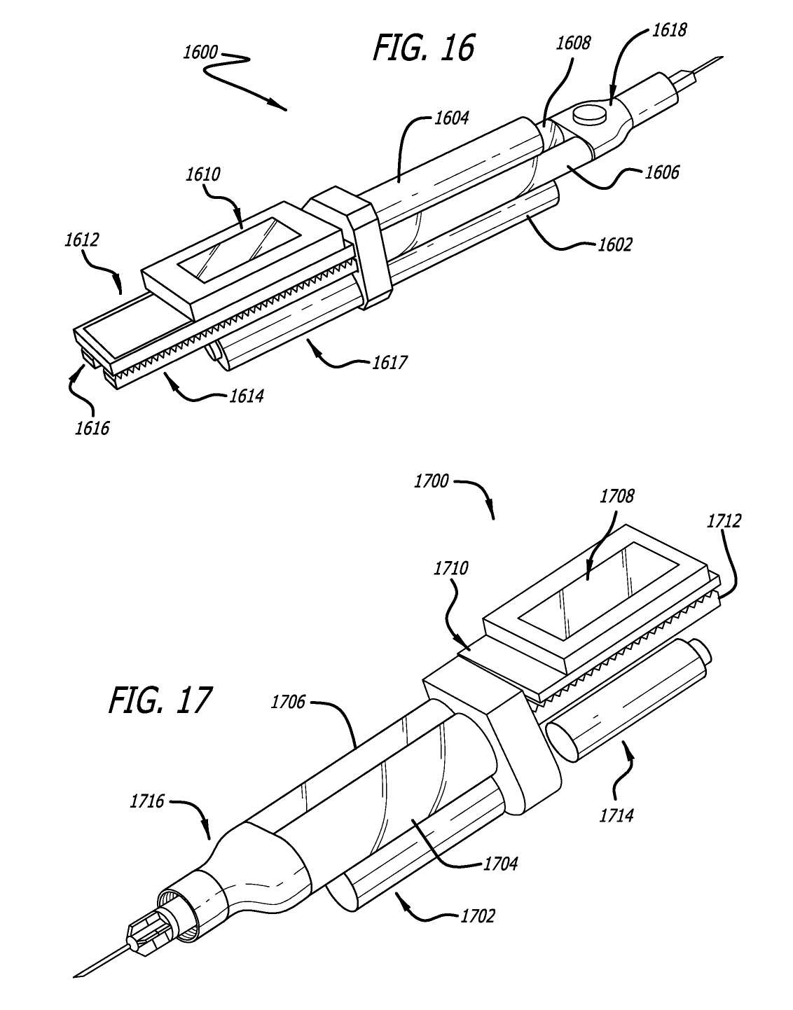

In another example, as illustrated in FIG. 16, components of injection device 1600 are configured such that configuration of injection device 1600 has a front section having a diamond shaped cross section based on the positions of motors 1602 and 1604 and cartridges 1606 and 1608. This diamond-shaped cross section may provide improved ergonomics. In this example configuration, injection device 1600 also includes: (a) display device 1610; (b) circuit board 1612; (c) first rack 1614; (d) second rack 1616; (e) first battery 1617; (f) second battery (not shown) positioned under the rack second rack 1616; and (g) mixing unit 1618. In this example, the cross section of injection device 1600 is fairly consistent along its length.

In another example, the components of injection device 10 are configured such that the weight of the injection device 10 is effectively balanced by positioning the motor(s) of the injection device 10 in the front section of injection device 10. For example, as illustrated in FIG. 17, single motor 1702 of injection device 10 is positioned under cartridges 1704 and 1706. In this example, the front section of injection device 1700 has a substantially triangular cross section, which may be beneficial for gripping purposes. Injection device 1700 of FIG. 17 also includes: (a) display device 1708; (b) circuit board 1710; (c) first rack 1712; (d) second rack (not shown) positioned under the circuit board 1710; (e) battery 1714; and (f) mixing unit 1716.

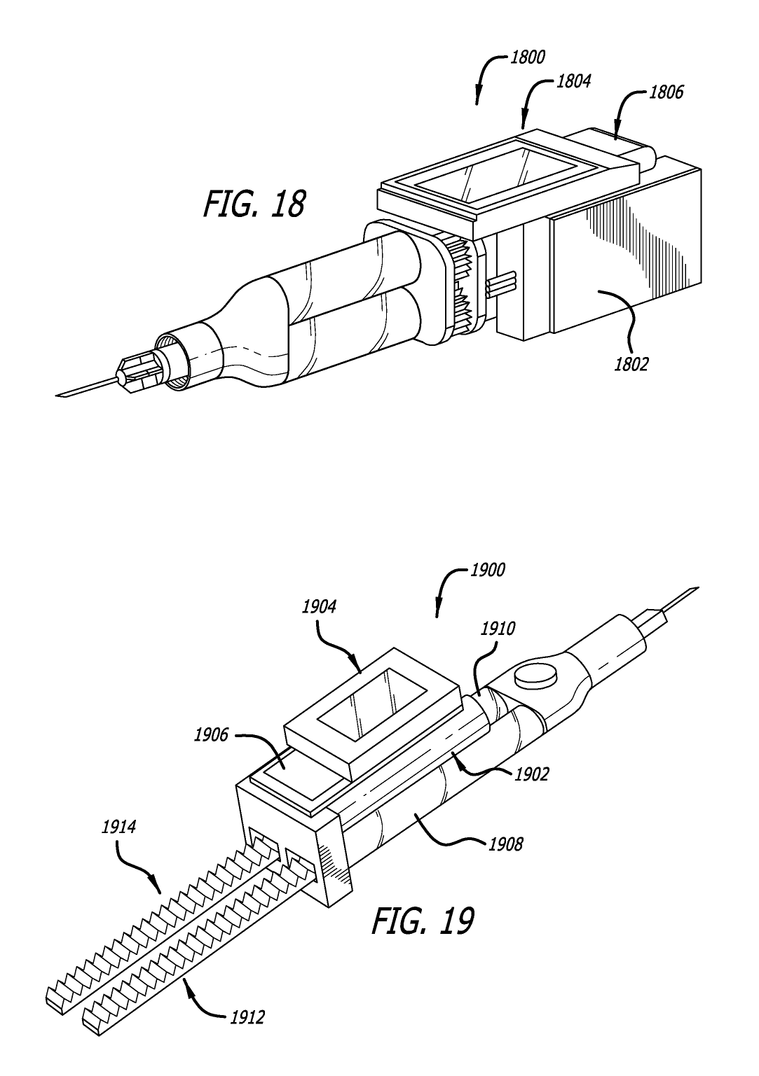

In one embodiment, components of injection device 10 are configured such that injection device 10 is configured to allow for potentially unobstructed viewing of cartridges by positioning most of the components of injection device 10 in the rear section of injection device 10. For example, the following components of injection device 1800 illustrated in FIG. 18 are positioned in the rear section: (a) battery 1802; (b) first motor (not shown); (b) second motor (not shown); (c) first rack (not shown); (d) second rack (not shown); (e) display device 1804; and (i) circuit board 1806. In this example, injection device 1800 is rear heavy, and allows for a narrow cross section at the position where a user's fingers would grip the injection device 1800.

In one embodiment, components of injection device 10 are configured such that injection device 10 has a compact front section. For example, the following components of injection device 1900 illustrated in FIG. 19 are positioned in the front section of injection device 1900: (a) batteries (not shown); (b) first motor 1902; (c) second motor (not shown); (d) display device 1904; (e) circuit board 1906; (f) first cartridge 1908; and (g) second cartridge 1910. In this example, injection device 1900 also includes racks 1912 and 1914. In this example, the configuration of injection device 1900 provides a large cross section at the position where a user's fingers would grip the injection device 1900.

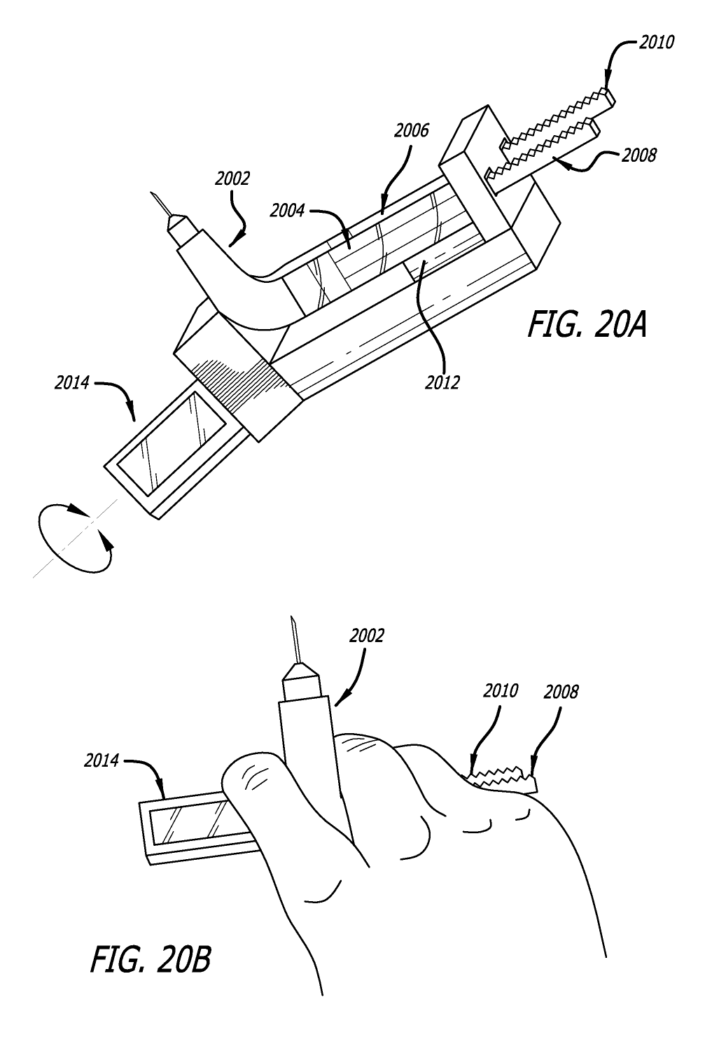

In one embodiment, the components of injection device 10 are configured such that injection device 10 enables a user to manipulate injection device 10 similar to an existing needle and cartridge device. For example, the following components of injection device 2000 of FIGS. 20A and 20B are arranged such that a user may manipulate the injection device 2000 similar to an existing needle and cartridge device: (a) mixing unit 2002; (b) first cartridge 2004; (c) second cartridge 2006; (d) first rack 2008; (e) second rack 2010; (f) first motor 2012; (g) second motor (not shown); and (h) display device 2014. In one embodiment, display device 2014 may be rotated for both right handed and left handed users.

In one embodiment, the input devices include at least one sensor. For example, injection device 10 may include a cartridge inserted sensor. Using the cartridge inserted sensor, the injection device may detect whether at least one cartridge is inserted in the cartridge housing. The cartridge inserted sensor may prevent the injection device from attempting to perform an injection without cartridge(s) properly loaded. In one embodiment, the injection device includes a home sensor. Using the home sensor, the injection device may detect whether the injection device is in a home state.

In one embodiment, injection device 10 includes at least one motor driver. In one embodiment, the motor driver communicates with both the processor and the motor(s). The motor driver may provide the systems necessary to control the operation of the motor(s). In one embodiment, using input from sensors and encoders, the processor directs the motor(s) through the motor driver, which in turn may control the extension of the plunger and thus the injection.

In addition, injection device 10 may include a power system. For example, injection device 10 may house at least one battery, or other power source (e.g., a rechargeable battery or a fuel cell). In one embodiment, the battery provides power to the control system. The battery may be connected to the control system in any suitable manner. For example, the battery may be permanently connected, e.g., soldered, or may be connected through a connector. In the later case, a door may be provided in the injection device, which may allow access to the battery for removal and replacement.

In addition, injection device 10 may include a battery charger. The battery charger may be capable of charging the at least one battery when connected to an external source of electricity. For example, the injector device may include a connector, which may allow the injector device to connect to a source of electrical power, such a standard 120 or 240 V AC power source. Of course, the injector device need not connect to such a power source directly. Rather the injector device may connect to a power adaptor or supply system, which may in turn connect to the primary power source. In addition, any suitable connector may be provided, e.g., in the body of the injection device, for connection to the external power source.

In one embodiment, the injection device includes disposable components. In one embodiment, the disposable components include anything that may come in contact with the injectable fluids (wet components). The disposable components may also include anything which is integral to the function of the wet components. For example, the disposable components may include a needle, syringes (filled with e.g., HA and PBS), plungers, o-rings, tubing, housings, fittings and/or seals.

In one embodiment, the injection device includes durable components which include components intended to be reused between patients. Therefore, in one embodiment, the injection device is easily cleaned. In one embodiment, the durable components include the drive unit, battery or batteries, the user interface, the printed circuit boards and any necessary electrical connections, the disposable retention mechanism for locking disposable and durable components together, and/or housings (e.g., lids, doors, slides, etc.).

The disposable components can be loaded into the durable components in any suitable way. For example, the disposable components can be loaded into the durable components employing slide in (slot) loading, drop in (shotgun) loading, and clip in loading, or any combination of these methods.

In the preceding specification, the present disclosure has been described with reference to specific example embodiments thereof. It will, however, be evident that various modifications and changes may be made thereunto without departing from the broader spirit and scope of the present disclosure. The description and drawings are accordingly to be regarded in an illustrative rather than restrictive sense.

The terms “a,” “an,” “the” and similar referents used in the context of describing the disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

In closing, it is to be understood that the embodiments of the disclosure disclosed herein are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

What is claimed is:

1. A dermal filler injection device comprising:
at least one processor;
at least one input device operatively coupled to the processor;
a first cartridge defining a first chamber configured to contain a first injectable fluid;
a second cartridge defining a second chamber configured to contain a second injectable fluid;
a drive unit operatively coupled to the processor;
a mixing unit configured to mix the first injectable fluid and the second injectable fluid; and
at least one memory device storing instructions which when executed by the at least one processor, causes the at least one processor, in cooperation with the at least one input device, the first cartridge, the second cartridge, the drive unit and the mixing unit, to:
(a) select a dilution ratio of the first injectable liquid and the second injectable liquid;
(b) based on the selected dilution ratio, produce an injectable mixed fluid by diluting the first injectable liquid with the second injectable liquid; and
(c) extrude the injectable mixed fluid.

2. The injection device of claim 1, wherein the first injectable fluid includes a dermal filler.

3. The injection device of claim 2, wherein the dermal filler is a hyaluronic acid-based dermal filler.

4. The injection device of claim 1, wherein the second injectable fluid includes a phosphate buffered saline.

5. The injection device of claim 1, wherein the drive unit includes:
(a) a plurality of gear motors; and
(b) a plurality of racks operatively coupled to the gear motors, the plurality of racks being operatively engaged with a plurality of plungers.

6. The injection device of claim 1, wherein the drive unit includes a pressure source and a pressure regulator.

7. The injection device of claim 1, wherein the instructions, when executed by the processor, causes the processor to, in cooperation with the at least one input device, select an injection rate.

8. The injection device of claim 6, wherein the instructions, when executed by the processor, causes the processor to extrude the injectable mixed fluid based on the selected injection rate.

9. The injection device of claim 1, wherein the instructions, when executed by the processor, causes the to processor, in cooperation with the at least one input device:
(a) select a first injection rate for the first injectable fluid; and
(b) select a second injection rate for the second injectable fluid.

10. A dermal filler injection device comprising:
at least one processor;
at least one input device operatively coupled to the processor;
a drive unit operatively coupled to the processor;
a mixing unit configured to mix the a first injectable fluid and a second injectable fluid; and
at least one memory device storing instructions which when executed by the at least one processor, causes the at least one processor, in cooperation with at least one cartridge which contains the first injectable fluid and the second injectable fluid, the at least one input device, a second cartridge, the drive unit and the mixing unit, to:
(a) select a dilution ratio of the first injectable liquid to the second injectable liquid;
(b) based on the selected dilution ratio, produce an injectable mixed fluid by diluting the first injectable liquid with the second injectable liquid; and
(c) extrude the injectable mixed fluid.

11. The injection device of claim 10, wherein the at least one cartridge includes a single cartridge.

12. The injection device of claim 10, wherein the instructions, when executed by processor, cause the processor to, in cooperation with the at least one input device, select an injection rate.

13. The injection device of claim 12, wherein the instructions, when executed by the processor, cause the processor to, in cooperation with the drive unit, extrude the injectable mixed fluid based on the selected injection rate.

14. The injection device of claim 10, wherein the instructions, when executed by the processor, cause the processor to, in cooperation with the at least one input device:
(a) select a first injection rate for the first injectable fluid; and
(b) select a second injection rate for the second injectable fluid.

15. An injection device comprising:
at least one processor;
at least one input device operatively coupled to the processor;
a first cartridge defining a first chamber configured to contain a first injectable fluid;
a second cartridge defining a second chamber configured to contain a second injectable fluid;
a drive unit operatively coupled to the processor;
a mixing unit configured to mix the first injectable fluid and the second injectable fluid; and
at least one memory device storing instructions which when executed by the at least one processor, causes the at least one processor, in cooperation with the at least one input device, the first cartridge, the second cartridge, the drive unit and the mixing unit, to:
(a) for the first injectable fluid, select a first injection rate;
(b) for the second injectable fluid, select a second injection rate;
(c) based on the selected first injection rate and the selected second injection rate, produce an injectable mixed fluid by diluting the first injectable liquid with the second injectable liquid; and
(d) extrude the injectable mixed fluid.

16. The injection device of claim 15, wherein the instructions, when executed by the processor, cause the processor to:
(a) determine an third injection rate for the injectable mixed fluid based on the selected first injection rate and the selected second injection rate; and
(b) cause a display device to display the third injection rate.
